(19) United States
(12) Patent Application Publication
(10) Pub. No.: US 2007/0251526 A1
(43) Pub. Date: Nov. 1, 2007

(54) Treatment of airflow

(76) Inventor: Marc Alan Zocher, Bainbridge Island, WA (US)

Correspondence Address: RG & ASSOCIATES, 1103 TWIN CREEKS, STE. 120, ALLEN, TX, 75013, US

(21) Appl. No.: 11/796,906

(22) Filed: Apr. 30, 2007

Related U.S. Application Data
(60) Provisional application No. 60/746,011, filed on Apr. 28, 2006.

(51) Int. Cl. A61N 5/06; A61M 16/00

(52) U.S. Cl. 128/204.18; 128/205.25; 607/88; 607/94

(57) ABSTRACT

An airflow treatment system is worn or carried by a user and irradiates an air flow with energy in the electromagnetic spectrum, such as UV light, before the air is inhaled by the user. The irradiated energy is of sufficient power and duration to substantially cleanse the air of airborne viruses and the like prior to inhalation. The system includes a light source with a power supply to generate the energy for irradiation. A face mask is worn by the individual so that outside air is drawn into the system, and the airflow passes through a airflow treatment chamber before it is inhaled by the user. The airflow is irradiated by the energy, such as UV light, while passing through the airflow treatment chamber such that airborne virus microorganisms in the airflow are substantially destroyed or rendered unharmful before the airflow is inhaled by the user.

### CROSS-REFERENCE TO RELATED APPLICATIONS

The present invention is related to and claims priority from U.S. provisional patent application Ser. No. 60/746,011, filed on Apr. 28, 2006, and titled System For Treatment Of Airflow, the entire contents of which are enclosed by reference herein.

### BACKGROUND

Vaccines are helpful in attacking airborne viruses and in preventing the spread of disease. Once a particular strain of virus is known, it is possible for a vaccine to be developed that is targeted at the particular virus. Persons who are inoculated with the vaccine are less likely to develop the ill effects of the virus, even after exposure to it. The public health care delivery system is often utilized to develop the vaccine and distribute it to health care centers for public inoculation programs. Although the cost of such programs can be high, the programs have been found to be effective at reducing the spread of disease and reducing suffering, and have been found to be an efficient use of public funds. Vaccines, however, must be developed for specific strains of airborne virus and are not useful against other, non-specific strains. Implementation of vaccine inoculation programs can require complicated and expensive infrastructure to develop, distribute, and administer. Moreover, the time to identify a virus, isolate it, and develop vaccines against it, means that a certain amount of disease and suffering will necessarily occur before inoculations can begin.

Limited environments, such as rooms in a building, can be protected against airborne viral infection by sealing off the environment to be protected from the outside world and treating circulating air within the sealed environment. Such rooms are often referred to as “clean rooms”. Circulating air can be treated by specially configured heating, ventilation, and air conditioning (HVAC) systems. For example, it is known that UV light can destroy viruses on HVAC components such as ducting and heat exchangers. The HVAC components can be bathed in ultraviolet (UV) light, thereby destroying airborne virus microorganisms. See, for example, U.S. Pat. No. 5,817,276 assigned to Steril-Aire. Unfortunately, protection of room environments does nothing to protect persons once they leave the protected environment and move about. Persons are only protected for the time they are within the clean room.

From the discussion above, it should be apparent that there is a need for protection against airborne virus infection that can be provided quickly and efficiently without a long development cycle and that provides protection for persons in a variety of environments as they move about. The present invention satisfies this need.

### SUMMARY

Embodiments of the invention pertain to a portable system that is worn or carried by a user and irradiates an air flow with energy in the electromagnetic spectrum, such as UV light, before the air is inhaled by the user. The irradiated energy is sufficient to substantially cleanse the air of airborne viruses and the like prior to inhalation. The system includes an airflow face mask worn by the individual so that outside air is drawn into the system, and the airflow passes through an airflow treatment chamber before it is inhaled by the user. The system includes a light source with a power supply to generate the energy for irradiation. The airflow is irradiated by the energy, such as UV light, while passing through the airflow treatment chamber such that airborne virus microorganisms in the airflow are substantially destroyed or rendered unharmful before the airflow is inhaled by the user. In this way, protection against airborne viruses is provided quickly and efficiently without a long development cycle and persons can remain protected as they move about in a variety of environments. The user is effectively protected by an external device at the most “downstream” portion of the viral exposure chain, so that viral exposure is defeated at the point of inhalation airflow, thus reducing the threat of viral infection independent of the strain or mutation of the virus.

In one aspect of the system, UV light for treatment is generated at the light source and is directed from the light source to the treatment chamber by fiber optics. The airflow treatment chamber is constructed so that air is drawn into the chamber and is irradiated sufficiently to destroy any airborne viruses. The irradiation can occur through an exposed end of the fiber optics used for directing UV light from the UV source, or through means such as a fiberglass mat in the treatment chamber or fiber optic rod or glass disk or terminus in the chamber, coupled to the fiber optics and configured so that the air flow in the airflow treatment chamber must pass by the rod or through the mat. In one embodiment, the mat can be provided as a weave of fibers through which airflow can pass. Alternatively, the mat can be provided as a perforated mat that irradiates the airflow as it passes through. The perforations can be provided, for example, by pulsing a laser to cut through an otherwise solid fiber optic mat and leave the openings in the mat. Other variations might be advantageous and can be utilized. For example, UV light may be better directed into the airflow if a flat fiber optic mat, glass disk, or similar terminus is coated or frosted on top and bottom surfaces for reduced transmissivity and then is perforated, such that airflow through the mat will necessarily receive UV radiation as it is emitted from the cylindrical holes (the “walls” of the perforations) and is absorbed by the passing airflow. The diameter of the perforations can be adjusted for desired UV radiation effect and airflow passage. Further, this flow may be through cylindrical holes that are bent or curved, thus increasing air turbidity in the passage through the UV radiation field to optimize viral transient time in the field as opposed to laminar airflow through a linear cylinder.

The breathing mask ensures that all air inhaled by the user will first pass through the airflow treatment chamber. The airflow treatment chamber can be located in a housing that is external to the mask, such as in a backpack or belt clip configuration, or the airflow chamber can comprise an elongated conduit. In any configuration, the airflow treatment chamber is adequate in size for the airflow to remain within and receive irradiation exposure for a time sufficient for satisfactory treatment before being inhaled by the user. Air flow into and out of the external treatment chamber can be controlled by valves that ensure sufficient treatment prior to inhalation.

In one embodiment of the invention an airflow treatment system carried on an individual person, comprises an airflow treatment chamber that receives environmental air that is drawn into the chamber as the person inhales, a breathing mask that is worn by the person and receives treated air from the airflow treatment chamber and directs the treated air to the person for inhalation, and a light treatment subsystem that treats the outside air in the airflow treatment chamber with radiated energy of the electromagnetic spectrum such that airborne viruses in the outside air are substantially rendered ineffective prior to inhalation by the person.

In another embodiment of the invention, an airflow treatment system comprises an airflow treatment chamber that receives environmental air and produces treated air, and a light treatment subsystem that treats the environmental air in the airflow treatment chamber with radiated energy of an electromagnetic spectrum such that at least one airborne virus in the environmental air is substantially rendered ineffective prior to inhalation of the treated air.

In a further embodiment of the invention, an airflow treatment system comprises an airflow treatment chamber that receives environmental air and produces treated air, and a light treatment subsystem that treats the environmental air in the airflow treatment chamber with radiated energy of an electromagnetic spectrum such that at least one airborne virus in the environmental air is substantially rendered ineffective prior to inhalation of the treated air.

In yet another embodiment of the invention, an airflow treatment system comprises an airflow treatment chamber that receives environmental air, a light treatment subsystem including a light source, wherein the light source produces ultraviolet energy that treats the environmental air in the airflow treatment chamber such that airborne viruses in the environmental air are substantially rendered ineffective prior to inhalation by a person, and a breathing mask containing the light treatment subsystem that directs the treated air to the person for inhalation.

In yet a further embodiment of the invention, a method for treating environmental air comprises receiving air in an airflow treatment chamber, treating the air by providing ultraviolet energy to the air, and providing the treated air to a person for inhalation.

Other features and advantages of the present invention should be apparent from the following description of the preferred embodiments, which illustrate, by way of example, the principles of the invention.

### BRIEF DESCRIPTION OF THE DRAWINGS

FIG. 1a illustrates a breathing mask and a light treatment subsystem according to an embodiment of the present invention;

FIG. 1b illustrates a fiber optic grid according to an embodiment of the present invention;

FIG. 1c illustrates a UV source according to an embodiment of the present invention;

FIG. 2a illustrates a face on view of the breathing mask according to an embodiment of the present invention;

FIG. 2b illustrates a face on view of the fiber optic grid according to an embodiment of the present invention;

FIG. 3 illustrates a fiber optic connector molded at a time of production according to an embodiment of the present invention;

FIG. 4 illustrates an exposed end of a fiber according to an embodiment of the present invention;

FIG. 5 illustrates a funnel or tunnel arrangement according to an embodiment of the present invention; and

FIG. 6 illustrates a block and a housing that the energy from an electromagnetic spectrum would feed into according to an embodiment of the present invention.

### DETAILED DESCRIPTION

In the following description, for purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form without unnecessary details that are known to those skilled in the art so as to avoid unnecessary details that could obscure understanding of the present invention. These and other embodiments of the invention along with many of its advantages and features are described in more detail in conjunction with the text below and attached figures.

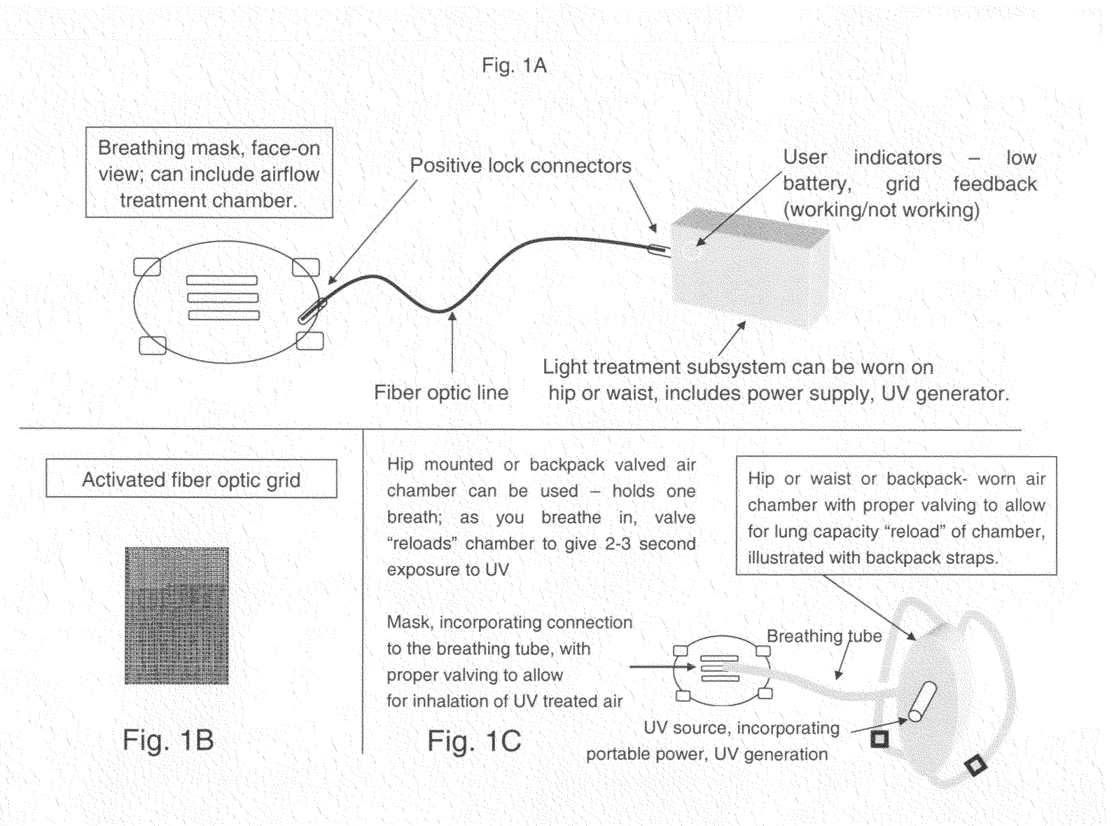

FIG. 1A illustrates construction details of an embodiment. The system includes an airflow breathing mask that is worn by the user. A light treatment subsystem treats incoming airflow before it is inhaled through the breathing mask. Between the light treatment subsystem and the breathing mask is an airflow treatment chamber in which the airflow is treated with energy from the electromagnetic spectrum, such as ultraviolet (UV) light. The breathing mask is worn on the face of the user and the light treatment subsystem is carried or worn on the body, and therefore the user need never be without the protection of the system, even as the person moves about through various environments.

The system can be constructed in a modular fashion, in that the breathing mask, light treatment subsystem, and airflow treatment chamber can be separated from the other components. Alternatively, components can be integrated. In FIG. 1A, for example, the system is constructed so that the airflow treatment chamber is located within the breathing mask, with sufficient protection from UV exposure to the dermal surface within the mask to prevent harm to the user. The light treatment subsystem can direct UV light to the breathing mask via a fiber optic line that extends from a connection at the light source to a connection at the breathing mask. In FIG. 1A, both ends of the fiber optic line attach by means of positive lock connectors that resist detachment, so that a deliberate motion is needed to disengage the fiber optic line from the breathing mask and from the UV light source.

The light source of the treatment subsystem includes a light generator and a power supply. The light generator produces the electromagnetic energy that is emitted into the airflow, such as UV light, and the power supply generates the electrical power to drive the light generator. The power supply can be provided as a battery pack or the like, with user indicators to show system status, such as battery charge, operational status, and the like. These user indicators may involve a feedback circuit to inform the user of a positive UV field through technologies such as photo sensors, RFID tags, and the like scaled to the device. The power supply includes a rechargeable battery and can be worn on the hip or waist of the user as well as in other areas.

The airflow treatment chamber can be constructed of a fiber optic weave or grid, so that spacing between fiber optic threads permits passage of the airflow. The fiber optic weave or grid is illustrated in FIG. 1B. Dimensions of the grid will be selected in accordance with the size of the breathing mask and with the extent needed for proper treatment of the airflow and for allowances of proper airflow velocity for breathing. An alternate design illustrated in FIG. 1C shows an airflow treatment chamber external to the breathing mask, receiving the electromagnetic energy from a fiber optic line or conduit from the light treatment subsystem. In the alternate design of FIG. 1C, the airflow treatment chamber can be hip-mounted or placed in a backpack or the like, external to the breathing mask. A valved air chamber can be used, to contain one breath in the chamber, such that as the user breathes in (inhales), the valve “reloads” the treatment chamber and holds the air within it, such that the inhaled air can remain in the chamber for an extended time, such as for approximately 2-3 seconds, and be irradiated, thereby prolonging the exposure of the air to the UV energy. An airflow line or breathing tube directs the airflow from the treatment chamber to the breathing mask for inhalation by the user.

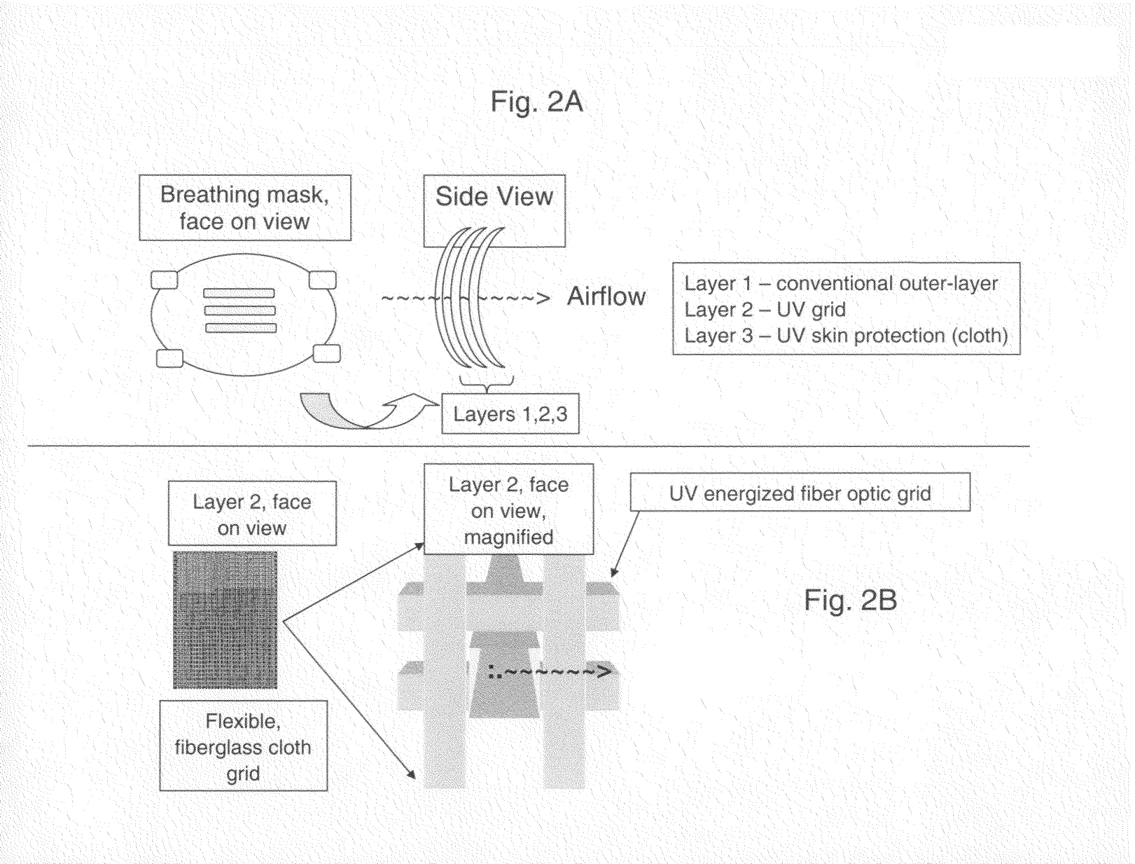

FIGS. 2A and 2B show details of an example of the fiber mat construction. In side view, FIG. 2A shows that the mat includes a conventional outer layer, a UV mat or grid layer, and a UV skin protection layer. All layers permit the passage of airflow. The conventional outer layer provides a particulate filtration function and exposes a durable outer surface to the environment. The middle UV grid layer directs the electromagnetic radiation into the airflow for treatment. That is, the UV energy is emitted from the middle layer. The inner UV skin protection layer protects the skin of the user against prolonged exposure to the UV radiation from the middle layer and is preferably of cloth construction or other relatively soft material for greater user comfort while wearing the breathing mask.

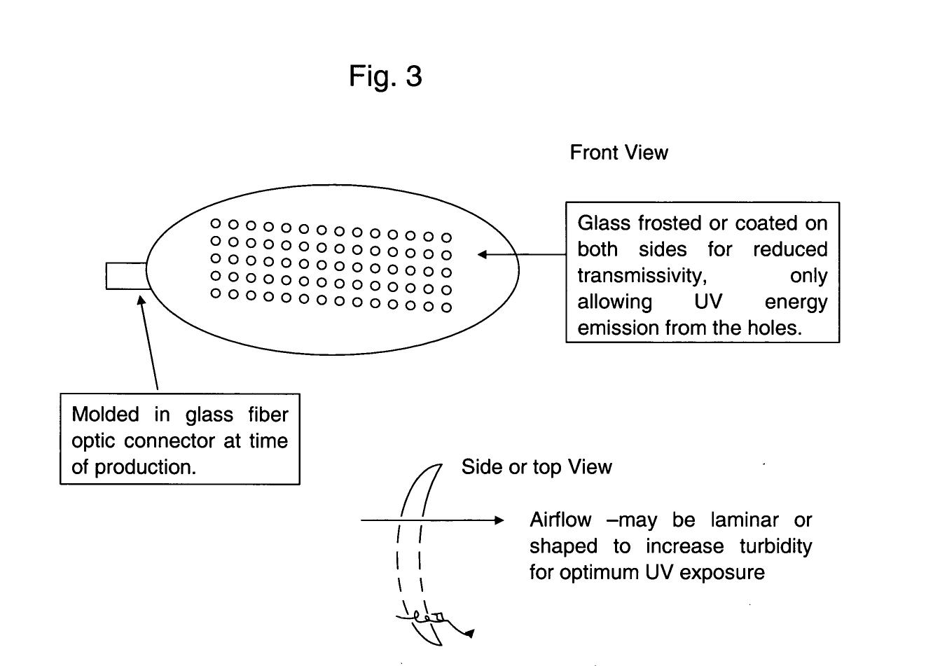

If the UV conducting fiber mat is provided as a weave, then FIG. 2B shows that it can have a multiple-ply construction, in a crisscross fashion. Alternatively, the fiber mat can be provided by a perforated construction that irradiates the airflow as it passes through. The perforated construction is illustrated in FIG. 3. The oval-shape structure in FIG. 3 is a perforated mat and is placed in the airflow. For example, the perforated mat can be placed in the airflow treatment chamber, and can comprise the middle layer of the breathing mask. Alternatively, the perforated mat can be placed in a breathing tube, such as the tube illustrated in FIG. 1B, or can be placed in an airflow treatment chamber, such as within the backpack of the configuration illustrated in FIG. 1B.

In other embodiments, an element or block with a chamber can receive the environmental air and trap or stall the air. Such air can be exposed to the energy for a longer period of time and allow a longer inhalation time. Further, an element or block with multiple chambers can receive and treat the environmental air in one chamber and trap or stall the treated air in another chamber so as to allow a longer inhalation time.

The perforations of the FIG. 3 mat can be provided, for example, by pulsing a laser to cut through an otherwise solid fiber optic mat, leaving behind the openings in the mat. To encourage the maximum amount of light energy into the airflow, the fiber material can be frosted or coated for reduced transmissivity, except that maximum emission of the UV energy will be emitted from the cut or perforated areas. In this way, the airflow through the mat will necessarily receive UV radiation that is emitted from the cylindrical holes or “walls” of the perforations, and the radiation will be absorbed by the passing airflow. The diameter of the perforations can be adjusted by varying the pulsing of the laser to increase or decrease the diameter of the perforations for controlling desired UV radiation effect and airflow passage. A further operational advantage of this configuration is the bi-directional air cleansing of both inhalation and exhalation of air through this UV layer. Since treatment of the air is non-specific to direction, a user that may have a previous viral infection may be non-symptomatic and unaware of the increased danger this poses to individuals within proximity to the wearer of this device. When the device is worn, this person will reduce the risk of transmission of viral material to others within the environment since exhaled air would also be treated. The connector in FIG. 3 and as described or depicted herein can be glass, quartz, Teflon, or other UV transmitting material.

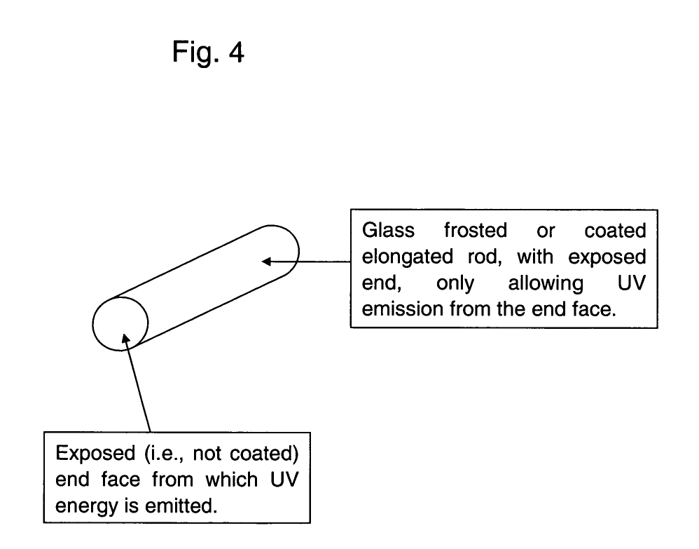

FIG. 4 shows another alternate configuration, in which irradiation of the airflow occurs through an exposed end of the fiber used for directing UV light from the UV source. The fiber is configured so that the exposed end of the fiber extends into the flow of air, such that the air must pass by the exposed fiber end prior to inhalation. In this way, the electromagnetic energy (e.g. UV energy) is radiated into the airflow with sufficient exposure to cleanse the airflow. The rod end can be placed in the breathing tube or in the airflow treatment chamber, whether that chamber is incorporated into the breathing mask (FIG. 1A) or on an external mounting, such as a backpack worn by the user (FIG. 1C). FIG. 4 shows the rod with a coating or frosting along its length and with an exposed (non-coated) end face, from which the UV energy is emitted.

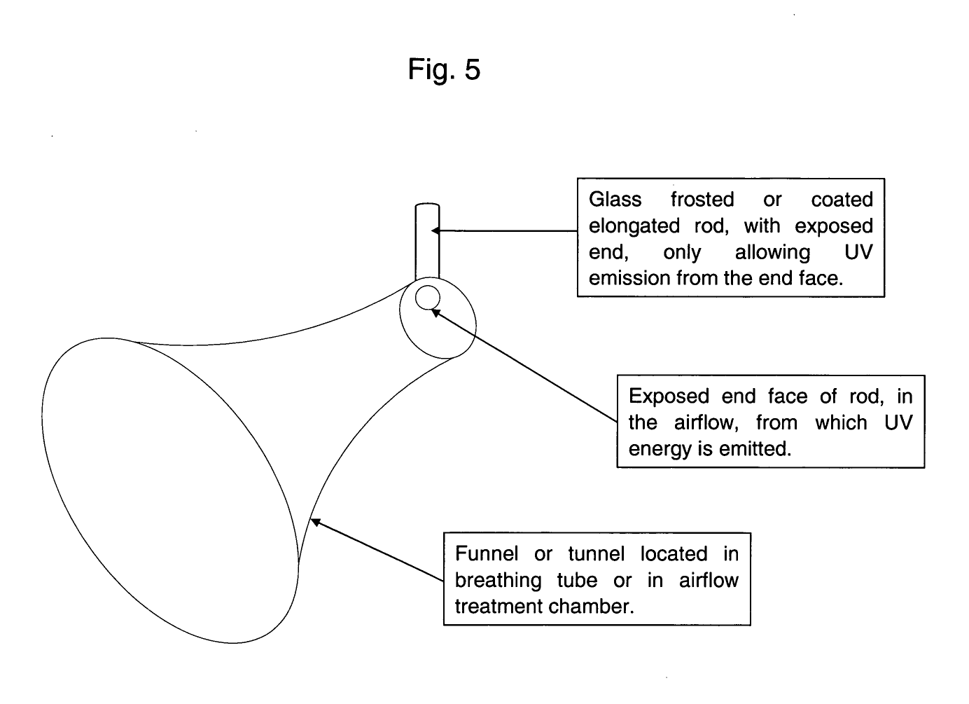

FIG. 5 shows yet another embodiment, in which the system includes a funnel or tunnel arrangement such that the airflow is directed into a funnel having a tapered diameter. As the airflow moves toward the user for inhalation, the airflow moves from a larger diameter end toward a smaller diameter end, whereupon the airflow is exposed to the electromagnetic radiation for treatment. Thus, the cross-sectional area of the airflow is concentrated toward the end of the funnel, so that the area of electromagnetic emission for treatment is reduced. FIG. 5 shows a rod such as the type illustrated in FIG. 4 that has been placed in the smaller diameter end of the funnel. The funnel configuration provides a smaller configuration for the system and reduces the surface area needed for radiation of the electromagnetic energy. The funnel arrangement illustrated in FIG. 5 can be placed in the breathing tube, such as the tube illustrated in FIG. 1C, or it can be placed in the airflow treatment chamber, whether that chamber is incorporated into the breathing mask (FIG. 1A) or on an external mounting, such as a backpack worn by the user (FIG. 1C).

As described in this document, the airflow treatment chamber can comprise any volume that is sealed off from the outer environment, in that air that is drawn into the chamber cannot exit and then be drawn back in. While the airflow is within the chamber, it is subjected to the electromagnetic energy with which it is cleansed. Thus, the treatment chamber can comprise the volume contained within the breathing mask illustrated in FIG. 1A, or can comprise a volume within the backpack configuration illustrated in FIG. 1C, or can comprise a breathing tube in which the air flows prior to inhalation by the user.

The light treatment subsystem can include any of the configurations described herein from which electromagnetic energy is emitted into the flow of air prior to inhalation. Thus, the light treatment subsystem can include the rod configuration, the fiber mat, the perforated mat, and the like having an optic or exposed surface from which the electromagnetic energy is emitted. The energy emitting optic of the light treatment subsystem can be placed at any location within the airflow where sufficient treatment of the air will occur. For example, the energy emitting optic can be located within the breathing mask, or the breathing tube, or the external airflow treatment chamber.

The breathing mask worn by the user must be sealed against the environment so that the user cannot inhale outside air, but can only inhale air that is delivered from the airflow treatment chamber. Thus, all of the air that is inhaled by the user who is wearing the breathing mask will first be treated with the electromagnetic energy from the light treatment subsystem as described herein.

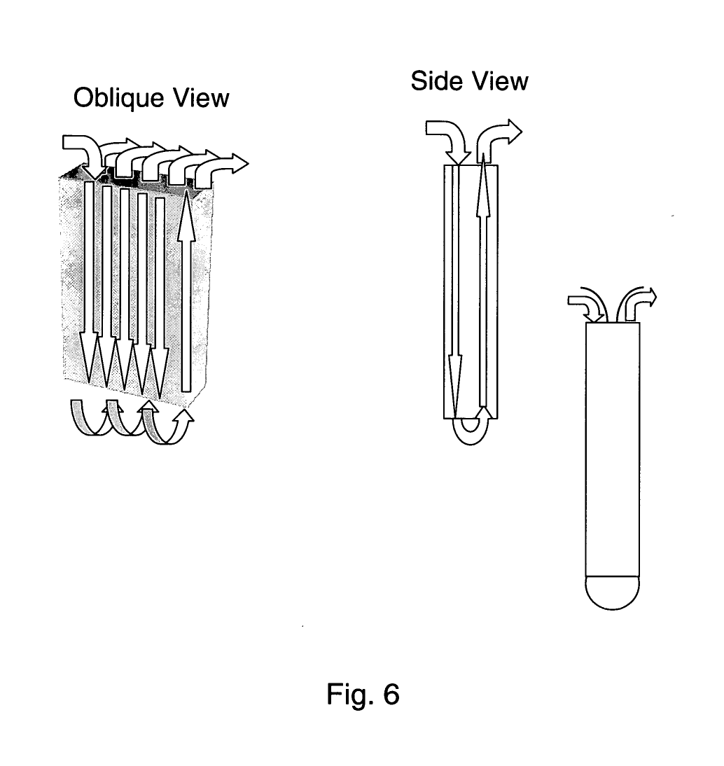

FIG. 6 shows yet a further embodiment, in which the system includes a block and a housing that the UV energy would feed into. The block is preferably comprised of quartz, glass, Teflon®, or other UV transmitting material designed with parallel hollow columns for airflow to allow for the longest possible airflow path in the least thickness of material. The direction of the arrows indicates the direction of the airflow. As can be seen from the side view, parallel hollow columns in two rows are included to allow double the contact time with the UV field. Air flows down one set of the columns and back up another. The final view is one of an opaque, sealed housing to house the UV block and to protect the skin from UV exposure. The housing, which may be made of plastic, metal, or other impervious material, is rounded at the bottom to promote airflow turbulence.

Having fully described several embodiments of the present invention, other equivalent or alternative methods of practicing the present invention will be apparent to those skilled in the art. For example, one or more of the functionalities described herein can be performed by one or more of the described or depicted elements. Also, a combination of elements may be used to permit various levels of transmissivity. For example, a combination of two or more transmissive materials such as glass, quartz, Teflon, and the like may be used. These and other embodiments as well as alternatives and equivalents to the described system will be recognizable to those of skill in the art after reading the description herein. The scope of the invention should not, therefore, be determined solely by reference to the above description, but instead should be determined with reference to the appended claims along with their full scope of equivalents and alternatives.

What is claimed is:

1. An airflow treatment system carried on an individual person, the system comprising:
an airflow treatment chamber that receives environmental air that is drawn into the chamber as the person inhales;
a breathing mask that is worn by the person and receives treated air from the airflow treatment chamber and directs the treated air to the person for inhalation; and
a light treatment subsystem that treats the outside air in the airflow treatment chamber with radiated energy of the electromagnetic spectrum such that airborne viruses in the outside air are substantially rendered ineffective prior to inhalation by the person.

2. The airflow treatment system as defined claim 1, wherein the radiated energy comprises ultraviolet (UV) energy.

3. The airflow treatment system as defined in claim 1, wherein the light treatment subsystem includes a light source that produces the electromagnetic energy.

4. The airflow treatment system as defined in claim 3, wherein the light source includes a radiation generator that emits the electromagnetic energy and a power supply that provides energy to the radiation generator.

5. The airflow treatment system as defined in claim 3, further including a fiber optic conduit that directs the electromagnetic energy from the light treatment subsystem so that it is radiated into the airflow treatment chamber.

6. The airflow treatment system as defined in claim 3, wherein the light treatment subsystem includes a fiber optic rod that emits the electromagnetic energy into the airflow.

7. The airflow treatment system as defined in claim 3, wherein the light treatment subsystem includes a fiber optic mat that emits the electromagnetic energy into the airflow.

8. The airflow treatment system as defined in claim 7, wherein the fiber optic mat is perforated.

9. The airflow treatment system as defined in claim 7, wherein the fiber optic mat is of reduced transmissivity.

10. The airflow treatment system as defined in claim 7, wherein the fiber optic mat comprises a fiber optic weave.

11. The airflow treatment system as defined in claim 3, wherein the light source includes a fiber optic that includes an area of reduced transmissivity and an exposed surface from which the electromagnetic radiation is emitted.

12. The airflow treatment system as defined in claim 1, wherein the airflow treatment chamber includes a funnel that directs the airflow into a reduced cross-section area.

13. An airflow treatment system, comprising:
an airflow treatment chamber that receives environmental air and produces treated air; and
a light treatment subsystem that treats the environmental air in the airflow treatment chamber with radiated energy of an electromagnetic spectrum such that at least one airborne virus in the environmental air is substantially rendered ineffective prior to inhalation of the treated air.

14. A method for treating environmental air, comprising:
receiving air in an airflow treatment chamber;
treating the air by providing ultraviolet energy to the air; and
providing the treated air to a person for inhalation.

15. The method as defined in claim 14 comprising providing the ultraviolet energy via a radiation generator.

16. The method as defined in claim 15 comprising providing energy to the radiation generator.

17. The method as defined in claim 14 comprising providing the ultraviolet energy via a fiber optic conduit.

18. The method as defined in claim 17 comprising directing the ultraviolet energy from a light treatment subsystem.

19. The method as defined in claim 18 comprising directing the ultraviolet energy from a fiber optic rod.

20. The method as defined in claim 18 comprising directing the ultraviolet energy from a fiber optic mat.
